(19) United States
(12) Patent Application Publication
(10) Pub. No.: US 2024/0142356 A1
(43) Pub. Date: May 2, 2024

(54) RECIPROCAL INJECTION DEVICE AND METHODS OF USING SAME

(71) Applicant: Merck Sharp & Dohme LLC, Rahway, NJ (US)

(72) Inventors: Guangli Hu, Summit, NJ (US); Steven Carl Persak, Morris Plains, NJ (US); Yongchao Su, Hillsborough, NJ (US); Edward Yeung, Whitestone, NY (US)

(73) Assignee: Merck Sharp & Dohme LLC, Rahway, NJ (US)

(21) Appl. No.: 18/490,075

(22) Filed: Oct. 19, 2023

Related U.S. Application Data
(60) Provisional application No. 63/419,824, filed on Oct. 27, 2022.

(51) Int. Cl. G01N 11/04; G01N 33/15

(52) U.S. Cl. CPC G01N 11/04; G01N 33/15

(57) ABSTRACT

In some embodiments, a reciprocal injection device includes a platform having a motor, a gearbox operatively coupled to the motor, an actuation slide driven by the motor, a pair of clamp fixtures for supporting two syringes, a pair of axially translatable syringe plunger fixtures, and an interface in communication with the platform and configured and arranged to actuate the motor.

### CROSS-REFERENCE TO RELATED APPLICATIONS

This application claims the benefit of U.S. Provisional Patent Application Ser. No. 63/419,824 filed Oct. 27, 2022, the entire contents of which are incorporated by reference herein.

### FIELD OF THE INVENTION

The present disclosure relates generally to devices and methods for testing and analyzing behavior of a pharmaceutical product, such as protein aggregation. More specifically, the present disclosure relates to devices and methods for simulating stress and extensional flow in syringes.

### BACKGROUND OF THE INVENTION

The quality and efficacy of pharmaceutical products may be affected by a host of environmental and manufacturing factors. For example, proteins are inherently sensitive to environmental factors that include hydrodynamic flow. Flow-induced protein remodeling is used in vivo and may trigger the aggregation of therapeutic proteins during manufacture. Currently, the relative importance of shear and extensional hydrodynamic flow fields to aggregation remains unclear. This is because conventional techniques rely on inducing protein by passing the fluid through a constriction, but do not utilize an electronic interface to accurately actuate the system for accurate, precise and repeatable results. Thus, it would be useful to simulate and test pharmaceutical products to test drug product stability and/or to pre-screen different primary containers with a drug product.

Thus, there exists a need for devices that improve upon and advance the methods of simulating and testing drug products.

### SUMMARY OF THE INVENTION

Described herein are reciprocal injection devices that offer the advantage of recirculation of both the shear state and the concentration cycling of formulations, such as protein-based formulations, as a result of the fluid flow through a narrowing and expanding channel that is the syringe-based setup. The reciprocal injection devices described herein provide a material sparing approach to conditioning various formulations, for example, high concentration protein formulations for subsequent testing and to assess any impact to product quality attributes.

Described herein are reciprocal injection devices. In some embodiments, the reciprocal injection device includes a platform having a motor, a gearbox operatively coupled to the motor, an actuation slide driven by the motor, a pair of clamp fixtures for supporting two syringes, and a pair of axially translatable syringe plunger fixtures; and an interface in communication with the platform and configured and arranged to actuate the motor.

In some examples, the platform includes a flat baseplate supporting the pair of clamp fixtures. In some examples, each of the pair of syringe plunger fixtures is independently movable via the motor. In some examples, the interface includes a display. In some examples, the display includes a touchscreen. In some examples, the interface is configured and arranged to allow a user to select at least one parameter relating to an injection. In some examples, the at least one parameter relating to an injection includes at least one of a syringe size, a plunger speed, a dwell time and a number of cycles. In some examples, the interface further includes an emergency stop button. In some examples, the interface and the platform are wirelessly connected or connected via a cable harness. In some examples, the interface further includes a status indicator light. In some examples, the interface further includes a memory for recording information relating to an injection. In some examples, a system includes the reciprocal injection device, two syringes and a needle connection directly coupling the two syringes with one another.

Also described herein are methods for testing a substance using the reciprocal injection devices described herein. In some embodiments, the method of testing a substance includes providing a reciprocal injection device including a platform having a motor, a gearbox operatively coupled to the motor, an actuation slide driven by the motor, a pair of clamp fixtures for supporting two syringes, and a pair of syringe plunger fixtures, and an interface in communication with the platform and configured and arranged to actuate the motor, positioning two syringes in the pair of clamp fixtures, and selecting a parameter on the interface to actuate the motor of the platform.

In some examples, selecting a parameter includes selecting at least one of a syringe size, a plunger speed, a dwell time and a number of cycles. In some examples, the method further includes placing the platform in an environmental chamber and placing the interface outside the environmental chamber. In some examples, the method further includes providing the interface with a memory and recording information relating to an injection in the memory. In some examples, the method further includes alerting a user to a status of an injection. In some examples, the method further includes wirelessly communicating information from the interface to the platform. In some examples, the method further includes providing a cable harness between the interface and the platform. In some examples, the method further includes injecting a substance from one of the two syringes by axially translating a corresponding one of the pair of syringe plunger fixtures. In some examples, the method further includes providing a needle connection to directly couple the two syringes with one another.

### BRIEF DESCRIPTION OF THE DRAWINGS

Various embodiments of the presently disclosed reciprocal injection device are disclosed herein with reference to the drawings, wherein:

FIG. 1 is a schematic illustration of one example of a syringe.

FIG. 2 is a schematic perspective view of one example of a reciprocal injection device.

FIG. 3 is a detailed view of one example of a platform of the reciprocal injection device.

FIG. 4 is a detailed view of a needle connection for use with the reciprocal injection device of FIG. 2.

FIG. 5 is a detailed view of an interface of the reciprocal injection device.

Various embodiments will now be described with reference to the appended drawings. It is to be appreciated that these drawings depict only some embodiments of the disclosure and are therefore not to be considered limiting of its scope.

### DETAILED DESCRIPTION OF THE INVENTION

Despite the various improvements that have been made to drug product delivery and storage, conventional device and methods suffer from some shortcomings, such as uncertainty of the relative importance of shear and extensional hydrodynamic flow fields to aggregation, as discussed above.

Therefore, there is a need for further improvements to the devices and methods used to help facilitate proper storage and delivery of pharmaceutical products. Among other advantages, the present disclosure may address one or more of these needs.

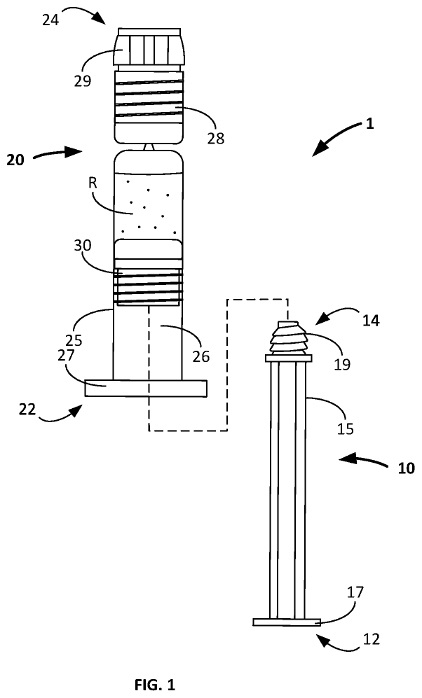

Reference is now made to FIG. 1, which shows a schematic illustration of a syringe 1. Syringe 1 may be a pre-filled syringe that generally comprises two main portions, a plunger rod 10 and a barrel 20. In one embodiment, barrel 20 contains a luer lock. In another embodiment, barrel 20 contains a stacked needle. Plunger rod 10 generally extends between a proximal end 12 and a distal end 14, and comprises an elongated piston 15 extending between a thumb press 17 and a coupler 19. In one embodiment, piston 15 has a cruciform cross-sectional shape. In one embodiment, thumb press 17 has a circular shape.

A cylindrical barrel 20 extends between proximal end 22 and distal end 24 and comprises a body 25 defining a lumen 26 for accepting a portion of plunger rod 10. As used herein, a “proximal” end means the end closer to a user and a “distal” end means the end away from the user. Body 25 further comprises a flange 27 adjacent proximal end 22, and a removable cap 29 mateable with a hub 28 adjacent distal end 24. Body 25 defines a reservoir “R” that holds a medicament, drug, saline, therapeutic protein(s), or other fluid or substance. An internally threaded stopper 13 is disposed inside lumen 26 of body 25. In one embodiment, stopper 13 is made of an elastomeric material such as natural rubber, synthetic rubber, thermoplastic elastomers, or combinations thereof, and includes an opening to receive and mate with coupler 19 of plunger rod 10 by advancing the plunger rod inside the barrel lumen 26 and rotating at least one of coupler 19 and stopper 13 relative to the other.

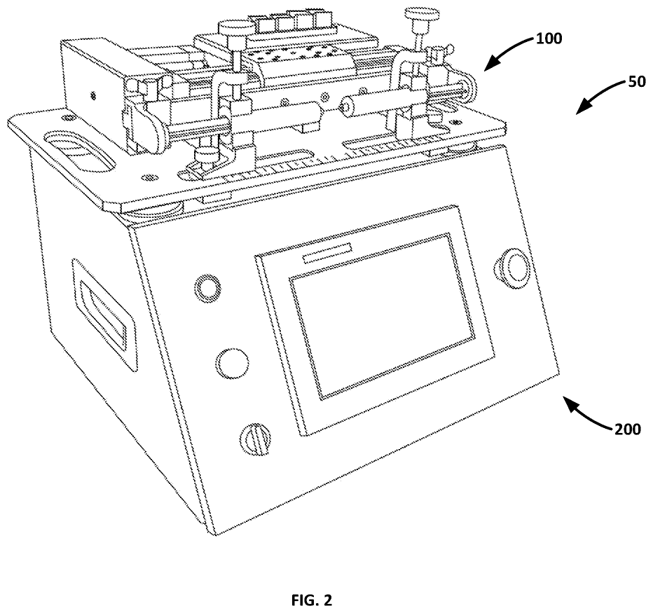

FIG. 2 illustrates one embodiment of a reciprocal injection device 50 that includes two main components: a work platform 100 and a corresponding human-machine interface 200. In the example shown, the work platform 100 is disposed on interface 200. In some examples, the work platform 100 and the interface 200 are unitarily formed. In some examples, the work platform 100 and the interface 200 are physically separate or separable. As will be discussed in greater detail, the work platform 100 and the interface 200 may be in communication with one another, and the connection may be a physical connection (e.g., via an ethernet connection, wire, or cabling) or a wireless connection (e.g., Wi-Fi, Bluetooth®, etc.).

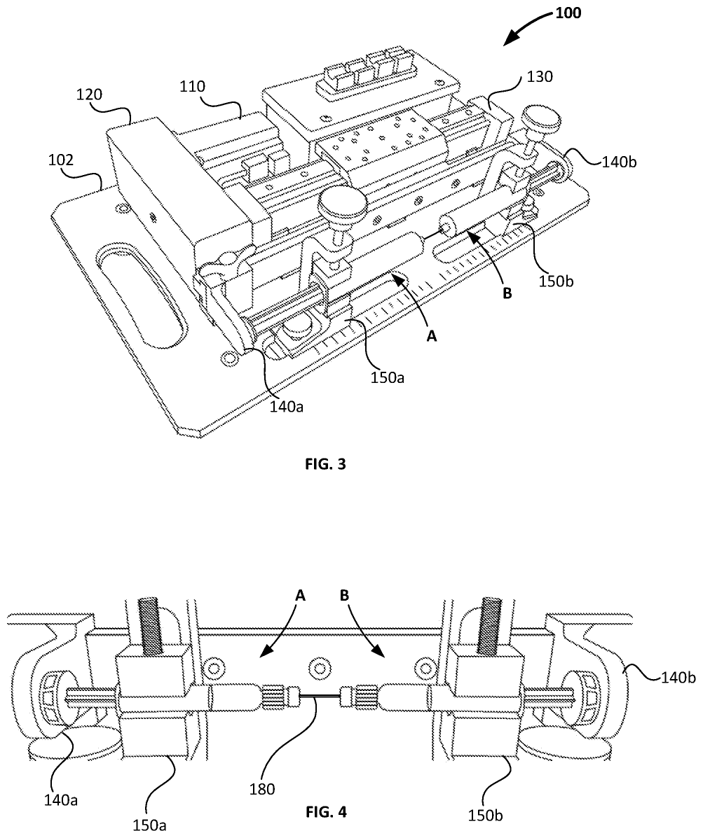

Turning to FIGS. 3-4, a detailed view of platform 100 is shown. In this example, platform 100 includes a flat baseplate 102 for supporting components of the platform (e.g., the motor, clamp fixtures, etc.). Baseplate 102 may have a flat upper surface to support these elements, and a flat lower surface to rest on the interface 200. In some examples, disposed on baseplate 102 is a motor 110, a gearbox 120 operatively coupled to motor 110, and a rodless actuator slide 130. A pair of adjustable clamp fixtures 150a, 150b are disposed on baseplate 102, the clamp fixtures being capable of receiving and/or supporting syringes of various sizes. In at least some examples, clamp fixtures 150a, 150b are disposed on opposite ends of baseplate 102 and spaced apart by a predetermined distance (e.g., between 6 and 24 inches). Adjacent each of the clamp fixtures 150a, 150b is a respective plunger fixture 140a, 140b that may be actuated by the motor. Specifically, each of the two plunger fixtures 140a, 140b may be independently driven or translated axially via motor 110. In some examples, the plunger fixtures 140a, 140b are capable of translating toward and/or away from one another.

In FIG. 3, two syringes, A and B are disposed in clamp fixtures 150a, 150b, respectively, the two syringes being of a similar size. It will be understood that the syringes may be mismatched in size, capacity and/or volume as desired, and that syringes ranging in volume from 6 mL to 20 mL are contemplated. Plunger fixture 140a may drive a plunger of syringe A, while plunger fixture 140b may drive the plunger of syringe B in an opposite direction. Turning to FIG. 4, a needle connection 180 may couple syringes A, B directly so that a fluid or substance can pass from one syringe to the other and vice versa. In some examples, needle connection 180 includes a dual hub luer-lock needle connection that is mateable with luer-locks of the two syringes, and the needle connection may be chosen based on the syringe sizes and needle gauge sizes.

Depending on the desired use, the platform 100 may actuate one of the plunger fixtures to depress a plunger and inject a substance from the reservoir of the syringe through the needle connection 180 and into the other syringe. This process may be reversed and/or repeated several times as desired, and the substance may flow through passages of predetermined diameters (e.g., from a large diameter to a smaller diameter or vice versa) to test the effects of shear and extensional hydrodynamic flow fields to aggregation.

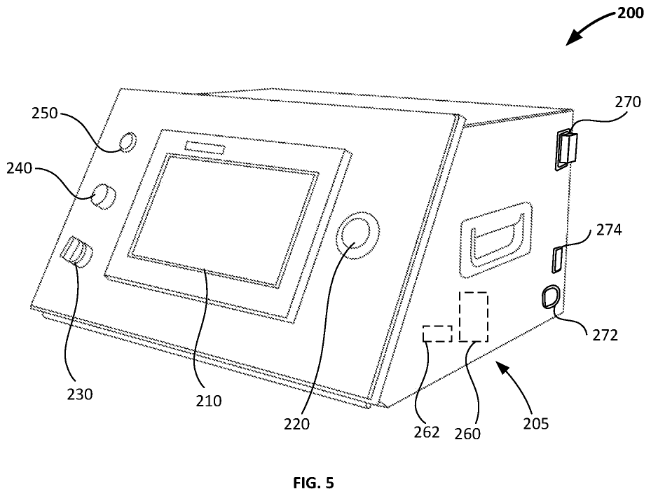

FIG. 5 shows details of a human-machine interface 200 that may be used to control platform 100. Interface 200 may generally include a rigid housing 205 comprising a plastic or metal (e.g., stainless steel), and display 210 to receive instructions from the user and relay information regarding the process of injection. In at least some examples, display 210 includes a 10-inch thin-film-transistor LCD touchscreen. Display 210 may alternatively include a touchscreen of a different size (e.g. 12 inches). Instead of a touchscreen, interface 200 may include separate display and data input components (e.g., a monitor, a keyboard, mouse, joystick, etc.). Interface 200 may also include an emergency stop button 220 to quickly shut down an injection process or halt progress of a syringe plunger fixture, a power switch 230 to turn on the machine or conserve power, and/or a reset button 240 to initialize the system and clear any values or parameters from a previous run. In some examples, an indicator 250 such as a status light indicator may be used to alert the user that an injection is in progress. Indicator 250 may be visual in the form of one or more lights, e.g., flashing, solid, white or colored lights (e.g., LEDs). Alternatively, or additionally, indicator 250 may incorporate an audible alert to the user, such as a beep or a chime to signal injection and/or completion. In the example shown, interface 200 includes a testing platform connector 270 disposed on the side of the interface, a power cord connector 272, an ethernet connection 274, and an internal programmable logic controller 260. In at least some examples, the physical connections (e.g., ethernet connection and/or testing platform connector) are long enough (e.g., greater than 2 feet, or greater than 4 feet) to allow the interface and the platform to be disposed in different locations. For example, the platform 100 may be disposed in a controlled environmental chamber at a selected temperature and/or pressure, while the interface 200 is disposed outside the environmental chamber. This may allow remote actuation of the platform from outside the chamber while studying thermal effects on a syringe product or substance. Interface 200 may further include additional processors and/or a memory 262 to manage and/or record data relating to an injection.

The interface may be configured and arranged to allow a user to select at least one parameter relating to an injection via display 210. This may include one or more parameters relating to the sizes, volumes and/or diameter of the syringe(s) A, B, the sizes and dimensions of the needle connection 180 syringe size, the plunger speed or plunger fixture speed, a dwell time (i.e., an amount of time for pause or rest between injections) and a number of cycles. For example, the interface 200 may accept the sizes of the syringes and the needle connector, accept properties relating to the substance in syringes A and/or B, select the speed of the plunger fixture (or the speed of the injection), and a dwell time of 5 seconds. This data may be stored on a memory within the interface or in a separate location (e.g., a cloud network) and may be transmitted for further analysis along with the sample. In at least some examples, the interface 200 and the platform 100 are wirelessly connected. Alternatively, a physical connection, such as a cable harness, ethernet cord or the like, may be used to transmit data from the interface 200 to instruct the platform 100 to inject a substance from syringe A to B or vice versa with a selected injection profile (e.g., a constant speed of 4 to 12 mm/sec (e.g., 8 mm/sec) or a non-linear speed of injection). The speed of plunger fixture 140a and plunger fixture 140b may be the same or different from one another. Additionally, a given plunger fixture 140a, 140b may be instructed to move at different speeds (e.g., alternating relatively fast and slow injections or gradually increasing or decreasing in magnitude) within the same test cycle.

In use, a method of testing a substance may include providing a reciprocal injection device including a platform having a motor, a gearbox operatively coupled to the motor, an actuation slide driven by the motor, a pair of clamp fixtures for supporting two syringes, and a pair of syringe plunger fixtures, and an interface in communication with the platform and configured and arranged to actuate the motor. The user may position two syringes within the pair of clamp fixtures and secure them so that they do not move when the plunger fixtures are actuated. A needle connector may be used to connect the two syringes together before or after they are placed in the clamp fixtures.

Once the platform is properly set up, the user may turn to the interface to select a parameter on the interface to actuate the motor of the platform. This may include toggling power switch 230 and selecting the appropriate parameter on display 210 including syringe size(s), plunger speed(s), a dwell time and/or a number of cycles. For example, the user may instruct the system to begin an injection from syringe A to syringe B at 8 mm/s, wait for 5 seconds, then inject from syringe B to syringe A at 10 seconds/mL and repeat the cycle 10 times. The dwell time may be selected to allow, for example, adequate cooling of the substance or drug product. In some examples, the delay between the transfer from syringe A to syringe B may be different then the delay between the transfer from syringe B to syringe A. Notably, the user may do this by placing the platform in an environmental chamber and placing the interface outside the environmental chamber and instructing the system from outside the chamber. The interface 200 may record information relating to an injection on the memory and save it locally or remotely. This information may be transmitted along with an experiment or sample identification number or tag for further analysis at a laboratory. While the reciprocal injection is in progress, the system may signal to the user through the display or the status indicator that they should not interrupt the process or enter the chamber.

The interface 200 will instruct the platform to perform the steps outlined by the user and the motor will drive the plunger fixtures, utilizing the gearbox to achieve the proper speed. Specifically, platform 100 contains all the mechanical parts that drive the system to produce reciprocal linear motion enabling for automated plunger movement. To create the linear motion the motor 110 may be connected to gearbox 120 to amplify and/or modulate the motor's motion and torque. The gearbox 120, in turn, may be connected to a rodless actuator slide 130 to convert the rotational motion from the motor and the gearbox to linear or axial movement of the plunger fixture(s). At the ends of the actuator slide 130 are syringe plunger fixtures 140a, 140b which translate the motion of the slide 130 to the syringes A, B. The syringe clamp fixtures 150a, 150b may secure the syringes in place to prevent movement of the syringe bodies, isolating only the plunger rod movement.

Using this system, an automated reciprocal syringe plunger movement for pumping fluid between two connected syringes is possible. The system may also be adapted for staked-in needles with appropriate connecting fixtures. The system and methods may serve as a drug product stability testing device and/or as a tool for pre-screening of different primary containers with a drug product. Specifically, the setup may be used to test the potential effects of hydrodynamic stress and spatiotemporal changes to multiphasic fluid concentration on pharmaceutical formulations by passing the fluid through from a larger to smaller diametrical opening. This may allow for studying the effects of hydrodynamic flow and shear stresses placed upon the fluid. The fluid medium and luer-lock needle connections can vary in different viscosities and gauges respectively, to further contrast the test setup and effects of hydrodynamic flow. In addition, this setup can also be utilized to differentiate primary container performances, such as the effects of silicone amounts, the silicone application, the effects of syringe transition regions from barrel to needle end, and/or to compare syringes from different vendors. Fluids that may be tested include, but are not limited to, medicaments, drugs, saline, therapeutic protein(s), therapeutic agents, pharmaceutical excipients, pharmaceutical formulations of biologics, compounds that have a therapeutic effect in or on an animal, and/or other substances.

It is to be understood that the embodiments described herein are merely illustrative of the principles and applications of the present disclosure. For example, the system may be battery-operated or remotely instructed via a tablet or a smartphone. Additionally, the system may include more than one motor or additional components to convert rotational movement to linear movement. The interface may also be connected to the platform in other ways so that information from the platform is relayed back to the interface. For example, the user may adjust the clamp fixture and the system can identify and record the size of the syringe based on the position of the clamp fixture. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments,

What is claimed is:

1. A reciprocal injection device, comprising:
a platform comprising:
a motor;
a gearbox operatively coupled to the motor;
an actuation slide driven by the motor;
a pair of clamp fixtures for supporting two syringes; and
a pair of axially translatable syringe plunger fixtures; and
an interface in communication with the platform and configured and arranged to actuate the motor.

2. The reciprocal injection device of claim 1, wherein the platform further comprises a flat baseplate supporting the pair of clamp fixtures.

3. The reciprocal injection device of claim 1, wherein each of the pair of syringe plunger fixtures is independently movable via the motor.

4. The reciprocal injection device of claim 1, wherein the interface comprises a display.

5. The reciprocal injection device of claim 4, wherein the display comprises a touchscreen.

6. The reciprocal injection device of claim 1, wherein the interface is configured and arranged to allow a user to select at least one parameter relating to an injection.

7. The reciprocal injection device of claim 6, wherein the at least one parameter relating to an injection includes at least one of a syringe size, a plunger speed, a dwell time and a number of cycles.

8. The reciprocal injection device of claim 1, wherein the interface further comprises an emergency stop button.

9. The reciprocal injection device of claim 1, wherein the interface and the platform are wirelessly connected.

10. The reciprocal injection device of claim 1, wherein the interface and the platform are connected via a cable harness.

11. The reciprocal injection device of claim 1, wherein the interface further comprises a status indicator light.

12. The reciprocal injection device of claim 1, wherein the interface further comprises a memory for recording information relating to an injection.

13. A system comprising:
the reciprocal injection device of claim 1;
two syringes; and
a needle connection directly coupling the two syringes with one another.

14. A method of testing a substance, comprising:
providing a reciprocal injection device including a platform having a motor, a gearbox operatively coupled to the motor, an actuation slide driven by the motor, a pair of clamp fixtures for supporting two syringes, a pair of syringe plunger fixtures, and an interface in communication with the platform and configured and arranged to actuate the motor;
positioning two syringes in the pair of clamp fixtures; and
selecting a parameter on the interface to actuate the motor of the platform.

15. The method of claim 14, wherein selecting a parameter comprises selecting at least one of a syringe size, a plunger speed, a dwell time and a number of cycles.

16. The method of claim 14, further comprising placing the platform in an environmental chamber and placing the interface outside the environmental chamber.

17. The method of claim 14, further comprising providing the interface with a memory and recording information relating to an injection in the memory.

18. The method of claim 14, further comprising alerting a user to a status of an injection.

19. The method of claim 14, further comprising wirelessly communicating information from the interface to the platform.

20. The method of claim 14, further comprising providing a cable harness between the interface and the platform.

21. The method of claim 14, further comprising injecting a substance from one of the two syringes by axially translating a corresponding one of the pair of syringe plunger fixtures.

22. The method of claim 14, further comprising providing a needle connection to directly couple the two syringes with one another.
